(19) United States
(12) Patent Application Publication
(10) Pub. No.: US 2019/0062239 A1
(43) Pub. Date: Feb. 28, 2019

(54) PROCESS AND APPARATUS FOR DUAL FEED PARA-XYLENE EXTRACTION

(71) Applicant: UOP LLC, Des Plaines, IL (US)

(72) Inventors: Linda S. Cheng, Highland Park, IL (US); Veronica G. Deak, Chicago, IL (US); Joseph A. Montalbano, Elmhurst, IL (US); Anton N. Mlinar, Vernon Hills, IL (US)

(21) Appl. No.: 15/691,037

(22) Filed: Aug. 30, 2017

(51) Int. Cl. C07C 7/10; B01D 11/04

(52) U.S. Cl. CPC C07C 7/10; B01D 11/0488; B01D 11/0492

(57) ABSTRACT

The present invention relates to a process and apparatus for dual feed para-xylene extraction heavy desorbent and light desorbent aromatics complex flow scheme. More specifically, the present invention relates to a process and apparatus for dual feed para-xylene extraction heavy desorbent and light desorbent aromatics complex flow scheme that takes advantage of the separation efficiency already achieved by toluene methylation or toluene disproportionation processes by sending the high concentration para-xylene to xylene feed to the corresponding concentration position in the para-xylene extraction unit. This enables further para-xylene separation and recovery that can be done with greater energy and capital efficiency.

### FIELD

The present invention relates to a process and apparatus for dual feed para-xylene extraction using either heavy desorbent or light desorbent in an aromatics complex flow scheme. More specifically, the present invention relates to a process and apparatus for dual feed para-xylene extraction heavy desorbent and light desorbent aromatics complex flow scheme that takes advantage of the above equilibrium para-xylene produced by the toluene methylation or toluene disproportionation processes by sending the high concentration para-xylene feed to the corresponding concentration position in the para-xylene extraction unit. This enables para-xylene separation and recovery that can be done with greater energy and capital efficiency.

### BACKGROUND

Para-xylene and meta-xylene are important raw materials in the chemical and fiber industries. Terephthalic acid derived from para-xylene is used to produce polyester fabrics and other articles which are in wide use today. One or a combination of adsorptive separation, crystallization and fractional distillation have been used to obtain these xylene isomers, with adsorptive separation capturing a great majority of the market share of newly constructed plants for the dominant para-xylene isomer.

Processes for adsorptive separation are widely described in the literature. For example, a general description directed to the recovery of para-xylene was presented at page 70 of the September 1970 edition of Chemical Engineering Progress (Vol. 66, No 9). There is a long history of available references describing useful adsorbents and desorbents, mechanical parts of a simulated moving-bed system including rotary valves for distributing liquid flows, the internals of the adsorbent chambers and control systems.

Energy requirements for the production of para-xylene from an aromatics complex are intensive, particularly in the PIX loop (para-xylene extraction, isomerization, and xylene fractionation) as the recycle traffic is high due to the equilibrium limit of xylene isomerization. Therefore, it's desirable to reduce the PIX loop traffic to increase the energy efficiency and capital requirement for para-xylene production.

One way to reduce PIX loop traffic is to feed concentrated sorbent, para-xylene, to the para-xylene extraction unit. Toluene methylation and toluene disproportionation processes achieve above equilibrium para-xylene to xylene production. The existing process flow scheme blends the above equilibrium para-xylene to xylene stream from toluene methylation or toluene disproportionation processes with the equilibrium para-xylene to xylene from isomerization, transalkylation, and fresh reformate feed before sending it to xylene fraction and subsequently to para-xylene extraction. This reduces the separation efficiency and increases energy and capital cost of the PIX loop.

### SUMMARY

The present disclosure describes a process and apparatus for a dual feed para-xylene extraction heavy desorbent and light desorbent aromatics complex flow scheme. A first embodiment of the invention is a process for para-xylene extraction, comprising passing a first feed stream comprising hydrocarbons to a para-xylene extraction unit, passing a second feed stream comprising hydrocarbons to a para-xylene extraction unit; passing a light desorbent to the para-xylene extraction unit, operating the para-xylene extraction unit under para-xylene extraction conditions, removing an extract from the para-xylene extraction unit; and removing a raffinate from the para-xylene extraction unit. A second embodiment of the invention is an apparatus for para-xylene extraction, comprising a para-xylene extraction unit having an upper portion, an intermediate portion, and a lower portion, a desorbent line comprising light desorbent in communication with the upper portion of the para-xylene extraction unit, a first feed line in communication with the lower portion of the para-xylene extraction unit, a second feed line in communication with the intermediate portion of the para-xylene extraction unit, an extract line in communication with the intermediate portion of the para-xylene extraction unit, and a raffinate line in communication with the lower portion of the para-xylene extraction unit.

In the foregoing, all temperatures are set forth in degrees Celsius and, all parts and percentages are by weight, unless otherwise indicated. Other objects, advantages and applications of the present invention will become apparent to those skilled in the art from the following detailed description and drawing. Additional objects, advantages and novel features of the examples will be set forth in part in the description which follows, and in part will become apparent to those skilled in the art upon examination of the following description and the accompanying drawing or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWING

The FIGURE illustrates an embodiment of the dual feed para-xylene extraction process and apparatus.

### Definitions

As used herein, the term “stream” can include various hydrocarbon molecules and other substances.

As used herein, the term “stream”, “feed”, “product”, “part” or “portion” can include various hydrocarbon molecules, such as straight-chain and branched alkanes, naphthenes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C1, C2, C3, Cn where “n” represents the number of carbon atoms in the one or more hydrocarbon molecules or the abbreviation may be used as an adjective for, e.g., non-aromatics or compounds. Similarly, aromatic compounds may be abbreviated A6, A7, A8, An where “n” represents the number of carbon atoms in the one or more aromatic molecules. Furthermore, a superscript “+” or “−” may be used with an abbreviated one or more hydrocarbons notation, e.g., C3+ or C3−, which is inclusive of the abbreviated one or more hydrocarbons. As an example, the abbreviation “C3+” means one or more hydrocarbon molecules of three or more carbon atoms.

As used herein, the term “unit” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 30%, preferably 70% and more preferably 90%, by mole, of a compound or class of compounds in a stream.

As depicted, process flow lines in the FIGURE can be referred to interchangeably as, e.g., lines, pipes, feeds, gases, products, discharges, parts, portions, or streams.

The term “communication” means that material flow is operatively permitted between enumerated components.

The term “predominantly” means a majority, suitably at least 50 mol % and preferably at least 60 mol %.

The term “passing” means that the material passes from a conduit or vessel to an object.

The term “light” desorbent is defined as anything boiling before C8 aromatics (i.e. is lighter than C8 aromatics). A light desorbent is typically toluene.

The term “heavy” desorbent is defined as anything boiling after C8 aromatics. A heavy desorbent is typically this is pDEB (para-diethylbenzene).

### DETAILED DESCRIPTION

The following detailed description is merely exemplary in nature and is not intended to limit the application and uses of the embodiment described. Furthermore, there is no intention to be bound by any theory presented in the preceding background or the following detailed description.

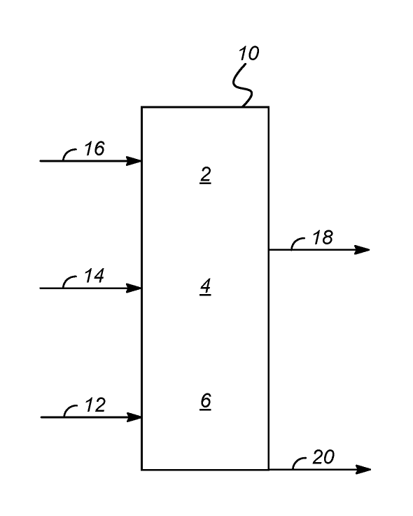

The description of the apparatus of this invention is presented with reference to the attached FIGURE. The FIG. 1s a simplified diagram of the preferred embodiment of this invention and is not intended as an undue limitation on the generally broad scope of the description provided herein and the appended claims. Certain hardware such as valves, pumps, compressors, heat exchangers, instrumentation and controls, have been omitted as not essential to a clear understanding of the invention. The use and application of this hardware is well within the skill of the art.

The various embodiments described herein relate to a dual feed para-xylene extraction process. As shown in the FIGURE, an apparatus 10 comprises of a para-xylene separation unit having two feeds. Para-xylene separation unit 10 is based on an adsorptive separation process. The para-xylene separation unit 10 comprises of a lower portion 6, an intermediate portion 4, and an upper portion 2. In the example shown in the FIGURE, the first feed 12 enters the para-xylene separation unit 10 in the lower portion 6. The first feed 12 is comprised predominantly by xylenes and ethylbenzene with minor amounts of C7 and C9 material and has a para-xylene to xylene ratio smaller than the second feed 14. However, in some embodiments, it is contemplated that the first feed 12 may enter the para-xylene separation unit 10 in another location, as long as it is below the location of the second feed 14.

As described above, the para-xylene separation unit 10 is based on an adsorptive separation process. Such adsorptive separation can recover over 99 wt-% pure para-xylene at high recovery per pass. Thus, the raffinate 20 from the para-xylene separation unit 10 is almost entirely depleted of para-xylene, to a level usually of less than 1 wt-%. The raffinate 20 may be sent to an alkylaromatics isomerization unit, where additional para-xylene is produced by reestablishing an equilibrium or near-equilibrium distribution of xylene isomers. Any ethylbenzene in the para-xylene separation unit raffinate 20 is either converted to additional xylenes or converted to benzene by dealkylation, depending upon the type of isomerization catalyst used. Separation of para-xylene from the other xylenes in the para-xylene separation unit 10 results in the formation of an extract stream 18 containing para-xylene.

In the example shown in the FIGURE, the second feed 14 enters the para-xylene separation unit 10 in the intermediate portion 4. The second feed 14 also comprises predominantly xylenes, but with a para-xylene to xylene ratio larger than of feed 12. However, in some embodiments, it is contemplated that the second feed 14 may enter the para-xylene separation unit 10 in another location, as long as it is above the location of the first feed 12. In the example shown in the FIGURE, the second feed stream is richer in para-xylenes to xylenes than the first feed stream. It is contemplated that the second feed stream may originate from a toluene methylation unit or a toluene disproportionation unit. If the feed stream originates from a toluene methylation unit, which may consist of a high temperature toluene methylation operating at about 500° C. to about 600° C. or a low temperature toluene methylation unit operating at about 200° C. to about 400° C.

A para-xylene depleted stream is withdrawn from para-xylene separation unit 10 as a raffinate stream 20 for further product recovery. The para-xylene depleted raffinate stream 20 may be sent to an isomerization unit. The raffinate stream 20 is removed from the bottom of the para-xylene separation unit 10. However, it is contemplated that the raffinate 20 may be removed from the para-xylene separation unit 10 at any location in the lower portion 6 of the para-xylene separation unit 10. An extract stream 18 is removed from the intermediate portion 4 of the para-xylene separation unit 10. The extract stream comprises a combination of xylenes, ethylbenzene, and desorbent, with a majority of the stream being comprised of para-xylene and desorbent.

The para-xylene separation unit 10 may operate under standard para-xylene separation unit conditions. For example, the para-xylene separation unit 10 may operate at a temperature of about 120° C. to about 150° C. when using a light desorbent. The para-xylene separation unit 10 may operate at a temperature of about 150° C. to about 180° C. when using a heavy desorbent.

While the invention has been described with what are presently considered the preferred embodiments, it is to be understood that the invention is not limited to the disclosed embodiments, but it is intended to cover various modifications and equivalent arrangements included within the scope of the appended claims.

### Specific Embodiments

While the following is described in conjunction with specific embodiments, it will be understood that this description is intended to illustrate and not limit the scope of the preceding description and the appended claims.

A first embodiment of the invention is a process for para-xylene extraction, comprising passing a first feed stream comprising hydrocarbons to a para-xylene extraction unit; passing a second feed stream comprising hydrocarbons to a para-xylene extraction unit; passing a light desorbent to the para-xylene extraction unit; operating the para-xylene extraction unit under para-xylene extraction conditions; removing an extract from the para-xylene extraction unit; and removing a raffinate from the para-xylene extraction unit. An embodiment of the invention is one, any or all of prior embodiments in this paragraph up through the first embodiment in this paragraph, wherein the first feed stream comprises xylenes and ethylbenzene. An embodiment of the invention is one, any or all of prior embodiments in this paragraph up through the first embodiment in this paragraph, wherein the second feed stream comprises xylenes and ethylbenzene. An embodiment of the invention is one, any or all of prior embodiments in this paragraph up through the first embodiment in this paragraph, wherein the second feed stream is richer in para-xylenes to xylenes than the first feed stream. The process of claim, wherein the second feed stream originates from a toluene methylation unit. An embodiment of the invention is one, any or all of prior embodiments in this paragraph up through the first embodiment in this paragraph, wherein the second feed stream originates from a toluene disproportionation unit. An embodiment of the invention is one, any or all of prior embodiments in this paragraph up through the first embodiment in this paragraph, wherein the para-xylene extraction conditions include a temperature of about 120° C. to about 150° C. An embodiment of the invention is one, any or all of prior embodiments in this paragraph up through the first embodiment in this paragraph, wherein the para-xylene extraction unit comprises a lower portion, an intermediate portion, and an upper portion. An embodiment of the invention is one, any or all of prior embodiments in this paragraph up through the first embodiment in this paragraph, wherein the first feed stream is passed to the lower portion of the para-xylene extraction unit. An embodiment of the invention is one, any or all of prior embodiments in this paragraph up through the first embodiment in this paragraph, wherein the second feed stream is passed to the intermediate portion of the para-xylene extraction unit.

A second embodiment of the invention is an apparatus for para-xylene extraction, comprising a para-xylene extraction unit having an upper portion, an intermediate portion, and a lower portion; a desorbent line comprising light desorbent in communication with the upper portion of the para-xylene extraction unit; a first feed line in communication with the lower portion of the para-xylene extraction unit; a second feed line in communication with the intermediate portion of the para-xylene extraction unit; an extract line in communication with the intermediate portion of the para-xylene extraction unit; and a raffinate line in communication with the lower portion of the para-xylene extraction unit. An embodiment of the invention is one, any or all of prior embodiments in this paragraph up through the second embodiment in this paragraph, wherein the para-xylene extraction unit operates at standard operating conditions. An embodiment of the invention is one, any or all of prior embodiments in this paragraph up through the second embodiment in this paragraph, wherein the para-xylene extraction unit operates at a temperature of about 120° C. to about 150° C. An embodiment of the invention is one, any or all of prior embodiments in this paragraph up through the second embodiment in this paragraph, wherein the second feed line originates from a toluene methylation unit. An embodiment of the invention is one, any or all of prior embodiments in this paragraph up through the second embodiment in this paragraph, wherein the second feed line originates from a toluene disproportionation unit.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for para-xylene extraction, comprising:
passing a first feed stream comprising hydrocarbons to a para-xylene extraction unit;
passing a second feed stream comprising hydrocarbons to a para-xylene extraction unit;
passing a light desorbent to the para-xylene extraction unit;
operating the para-xylene extraction unit under para-xylene extraction conditions;
removing an extract from the para-xylene extraction unit; and
removing a raffinate from the para-xylene extraction unit.

2. The process of claim 1, wherein the first feed stream comprises xylenes and ethylbenzene.

3. The process of claim 1, wherein the second feed stream comprises xylenes and ethylbenzene.

4. The process of claim 3, wherein the second feed stream is richer in para-xylenes to xylenes than the first feed stream.

5. The process of claim 1, wherein the second feed stream originates from a toluene methylation unit.

6. The process of claim 1, wherein the second feed stream originates from a toluene disproportionation unit.

7. The process of claim 1, wherein the para-xylene extraction conditions include a temperature of about 120° C. to about 150° C.

8. The process of claim 1, wherein the para-xylene extraction unit comprises a lower portion, an intermediate portion, and an upper portion.

9. The process of claim 8, wherein the first feed stream is passed to the lower portion of the para-xylene extraction unit.

10. The process of claim 8, wherein the second feed stream is passed to the intermediate portion of the para-xylene extraction unit.

11. An apparatus for para-xylene extraction, comprising:
a para-xylene extraction unit having an upper portion, an intermediate portion, and a lower portion;
a desorbent line comprising light desorbent in communication with the upper portion of the para-xylene extraction unit;
a first feed line in communication with the lower portion of the para-xylene extraction unit;
a second feed line in communication with the intermediate portion of the para-xylene extraction unit;
an extract line in communication with the intermediate portion of the para-xylene extraction unit; and
a raffinate line in communication with the lower portion of the para-xylene extraction unit.

12. The apparatus of claim 11, wherein the para-xylene extraction unit operates at standard operating conditions.

13. The apparatus of claim 11, wherein the para-xylene extraction unit operates at a temperature of about 120° C. to about 150° C.

14. The apparatus of claim 11, wherein the second feed line originates from a toluene methylation unit.

15. The apparatus of claim 11, wherein the second feed line originates from a toluene disproportionation unit.
